(19) United States
(12) Patent Application Publication
(10) Pub. No.: US 2006/0136062 A1
(43) Pub. Date: Jun. 22, 2006

(54) Height-and angle-adjustable motion disc implant

(76) Inventors: Alexandre DiNello, Cotuit, MA (US); Thomas M. DiMauro, Southboro, MA (US); Jeffrey Karl Sutton, Medway, MA (US); Michael O'Neil, West Bamstable, MA (US); Hassan Serhan, South Easton, MA (US); Michael Slivka, Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/015,927

(22) Filed: Dec. 17, 2004

(51) Int. Cl. A61F 2/44; A61F 2/30

(52) U.S. Cl. 623/17.14; 623/17.15; 623/18.12

(57) ABSTRACT

This invention relates to an intervertebral motion disc having a height adjustable endplate.

### BACKGROUND OF THE INVENTION

The leading cause of lower back pain arises from rupture or degeneration of lumbar intervertebral discs. Pain in the lower extremities is caused by the compression of spinal nerve roots by a bulging disc, while lower back pain is caused by collapse of the disc and by the adverse effects of articulation weight through a damaged, unstable vertebral joint. One proposed method of managing these problems is to remove the problematic disc and replace it with a prosthetic disc that allows for the natural motion between the adjacent vertebrae (“a motion disc”).

U.S. Pat. No. 4,759,766 (“Buttner-Janz”) discloses one such motion device comprising three components: an inferior endplate, a superior endplate, and a core having two articulation interfaces. Both the inferior and superior endplates have raised bosses with concave spherical articulation surfaces in the center. The core has convex surfaces on both the top and bottom that are surrounded by raised rims. The articulation surfaces of the core are designed to articulate with the articulation surfaces of the endplates.

Because articulating motion discs such as those described in Buttner-Janz seek to mimic the natural motion of the natural disc, it is desirable to place the disc at the precise location whereby the disc will have a center of rotation precisely equal to that of the natural disc. Accordingly, the device must be precisely placed at a predetermined spot during implantation in order mimic the natural center of rotation. However, it has been found that this is difficult to do in practice due to the fact that the prosthetic components are available only in discrete sizes varying by as much as one mm.

Although the surgeon can select a revision surgery to re-position the motion disc, such a surgery is costly and typically painful to the patient, and may include a risk of morbidity.

### SUMMARY OF THE INVENTION

The present inventors have noted that there may be a need to correct the height or angulation of a motion disc after the motion disc has been implanted. For example, because of the implantation is an inexact procedure, there may be times when the implanted disc is to tall or too short, or there is improper angulation. Accordingly, there may be a need to post-operatively correct the height or angle of the implant in order to adjust the height or angle to the new needs of the patient.

The present inventors have developed an intervertebral implant having an adjustable height and angle.

The implant of the present invention is advantageous because it can be inserted into the spine at a first height, and then adjusted to a second height to meet the needs of a particular patient.

In a first preferred embodiment, the height or angle of the implant is adjusted intra-operatively in order to fine tune the implant to the surgical needs of the patient. For example, such adjustment may allow precise tensioning of the annulus fibrosus, thereby preventing stenosis or ankylosis. In some embodiments, when the angle is adjusted, it is adjusted in either the coronal or saggital plane.

In a second preferred embodiment, the height of the implant is adjusted intra-operatively in order to fine tune the implant to the post-operative needs of the patient. This may occur if, for example, the prosthetic endplate sinks into the bony endplate.

Therefore, in accordance with the present invention, there is provided a prosthetic endplate in an intervertebral motion disc, the endplate comprising:

i) an outer plate comprising
an outer surface adapted for fixation to a first vertebral body,
an inner surface having a threaded recess, and
a body portion therebetween,

ii) an inner plate comprising:
an inner surface having a first articulation surface,
an outer surface, and
a body portion therebetween having a threaded lateral surface,
   wherein the threaded lateral surface of the body portion of the inner plate is mated with threaded recess of the outer plate.

### DESCRIPTION OF THE FIGURES

FIG. 1 is a cross-section of a motion disc of the present invention having a threadably-mated endplate capable of height adjustment.

FIG. 2 is a cross-section of a motion disc having a first rigid endplate having a moveable wedge therein, and a second flexible endplate.

FIG. 3 is a plan view of an endplate having a cam and pawl height adjustment mechanism.

FIG. 4 is a side view of a ratcheting thread form configured for free rotation in tension and one way rotation in compression.

FIG. 5a is a cross-section of a motion disc having a worm and pinion gear.

FIG. 5b is a top view of the worm and pinion gear of FIG. 2a.

FIG. 6a is a cross-section of a motion disc having a wedge for angle adjustment.

FIG. 6b is a top view of the lower plate of a motion disc having a wedge for angle adjustment.

FIG. 7 is a cross-section of a motion disc having a ratcheted wedge for angle adjustment.

FIG. 8a is a cross-section of a motion disc having a wedge for angle adjustment.

FIG. 8b is a top view of the lower plate of a motion disc having a wedge for angle adjustment.

FIGS. 9a-c show the insertion of motion discs for scoliosis correction.

FIGS. 10a-c show the insertion and tethering of motion discs for scoliosis correction.

FIGS. 11a-c show the insertion and tethering of motion discs for scoliosis correction.

FIGS. 12a-d show the insertion of cushion-type motion discs for scoliosis correction.

### DETAILED DESCRIPTION OF THE INVENTION

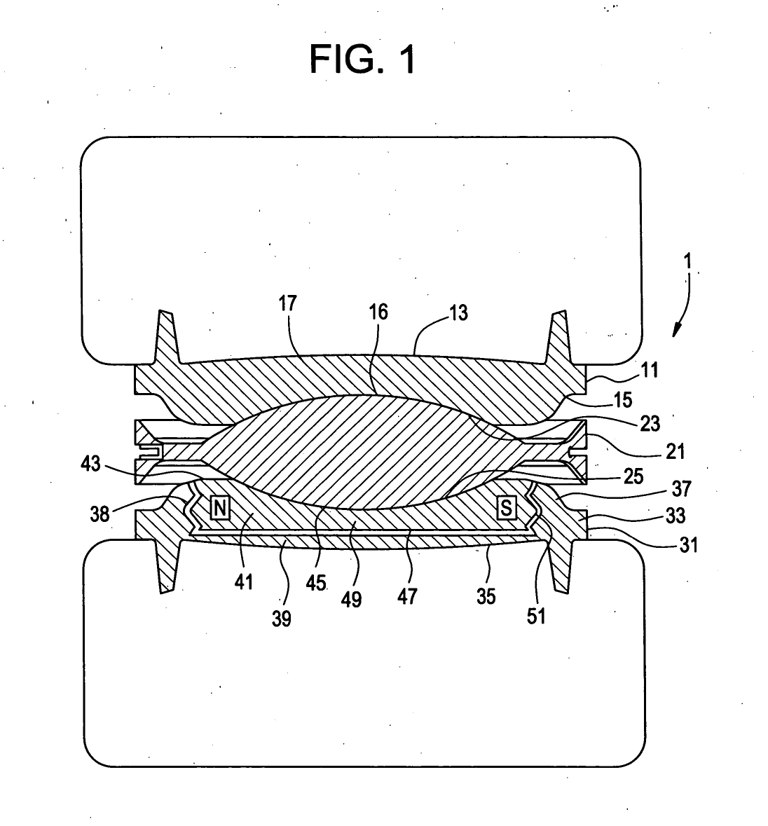

Now referring to FIG. 1, there is provided an intervertebral motion disc 1 comprising:

a) a first prosthetic vertebral endplate component 11 comprising:

i) an outer surface 13 adapted to mate with a first vertebral body,
ii) an inner surface 15 having a first articulation surface 16 suitable for supporting articulation motion thereon, and
iii) a body portion 17 connecting the inner and outer surfaces, and
   b) a core member component 21 comprising:
i) a first articulation surface 23 suitable for supporting articulation motion,
ii) a second articulation surface 25 suitable for supporting articulation motion,
   c) a second prosthetic vertebral endplate component 31 comprising:
i) an outer plate 33 comprising
   an outer surface 35 adapted for fixation to a second vertebral body,
   an inner surface 37 having a threaded recess 38, and
   a body portion 39 therebetween,
ii) an inner plate 41 containing a magnetic component and comprising:
   an inner surface 43 having a first articulation surface 45,
   an outer surface 47, and
   a body portion 49 therebetween having a threaded lateral surface 51,
      wherein the threaded lateral surface of the body portion of the magnetic inner plate is mated with threaded recess of the outer plate,
      wherein the first articulation surface of the core and first endplate are adapted to form a first articulation interface, and
      wherein the second articulation surface of the core and second endplate are adapted to form a second articulation interface.

Because the lower endplate 31 comprises a pair of threadably mated plates, and the outer plate thereof is anchored to the adjacent bone, it is apparent that inner plate can be rotated by a sufficiently strong applied force. Accordingly, when height adjustment is desired, an external magnetic force may be applied to the magnetic inner component of the lower endplate in a manner sufficient to cause rotation of the inner component. This rotation of the inner plate upon the threadform causes a change in height of the overall disc.

In this particular embodiment, the lower endplate comprises an inner plate having magnetic north N and south S poles. In use, a powerful external magnet (not shown) is placed near or on the patient's skin in the vicinity of the prosthetic endplate and rotated a predetermined amount in an orientation predetermined to causes rotation of the magnetic plate. The attractive-repulsive force produced between the external magnet and the magnetic nut is sufficient to effect rotation of the magnetic inner plate in a predetermined amount. As above, rotation of the magnetic inner plate causes relative movement of the inner plate in relation to the fixed outer plate, thereby adjusting the height of the motion disc.

These adjustments can be made based upon post-operative imaging results, as well as sensor-based determinations of distance or load indicating device mal-placement or movement and patient feedback.

The use of magnets to drive the height adjustment of implants is well known in the art. See, for example, U.S. Pat. No. 6,336,929 (“Justin”), the specification of which is hereby incorporated by reference in its entirety.

In some embodiments, the selected magnet comprises a rare earth metal. In other embodiments, the selected magnet is an electromagnet.

In the event that the actual surgery results in an acceptable positioning of the device, but a post-surgical shift occurs (for example, by adjacent level disc disease, trauma, injury or insufficient securement to the vertebral body) and produces a misalignment, the present invention can also be used to post-operatively adjust the height of the device.

The present invention may also allow the surgeon to adjust the relative positions of the components in order to optimize these relative positions based upon outcomes research that may appear in the literature after the disc has been implanted.

In some embodiments, the present invention further includes an implanted controller and an implanted sensor. These features may be easily adapted to provide automatic or closed loop adjustment of the center of rotation of the device without the need for physician or surgical intervention.

In some embodiments, sensor technology may be used to record the height of the disc as well as changes in height. For example, in some embodiments, the magnetic field created by the plate-based magnet of FIG. 1 could be detected by a Hall Effect sensor embedded near the outer surface of the upper endplate. The signals produced thereby may be sent to a MEMS-type controller-processor (also embedded near the inner surface of the plate). If adjustment were necessary, the controller/processor would then send a signal to a small motor (not shown) connected to or driving the adjustment screws to effect the desired adjustment.

In one aspect of the invention using Hall Effect sensors, there is provided in the endplate-endplate combination comprising at least one magnet in one of the endplates (the magnetic component), and at least one Hall Effect sensor in the other endplate (the sensor component). Preferably, the sensor component does not have any magnets thereon.

In some embodiments, height determination may be provided by the inclusion of radioopaque markers within two of the components within the disc.

In preferred embodiments, the prosthetic endplate (as opposed to a core component) is selected as the sensor component. This accommodates the need for robust circuitry needed to actuate the sensor and allows for thin film manufacturing techniques.

The motion disc component of the present invention can be, any prosthetic capable of restoring the natural motions of the intervertebral disc. In preferred embodiments, the motion disc is selected from the group consisting of an articulating disc, a cushion disc and a spring-based disc.

In some embodiments, the general structure of the articulating motion disc comprises:

a) a first prosthetic vertebral endplate comprising:

i) an outer surface adapted to mate with a first vertebral body,
ii) an inner surface having a first articulation surface,
iii) a body portion connecting the inner and outer surfaces,
   b) a second prosthetic vertebral endplate comprising:
i) an outer surface adapted to mate with a second vertebral body, and
ii) an inner surface comprising a first articulation surface,
   c) a core member comprising:
i) a first articulation surface adapted for articulation with the first articulation surface of the first endplate, and
ii) a second articulation surface adapted for articulation with the first articulation surface of the second endplate,
   wherein the core member is oriented to produce a first articulation interface between the first articulation surface of the first endplate and the first articulation surface of the core member, and a second articulation interface between the first articulation surface of the second endplate and the second articulation surface of the core member.

In some embodiments, the general structure of the articulating motion disc is a two piece design and comprises:

a) a first prosthetic vertebral endplate comprising:
i) an outer surface adapted to mate with a first vertebral body,
ii) an inner surface having a first articulation surface,
iii) a body portion connecting the inner and outer surfaces,

b) a second prosthetic vertebral endplate comprising:
i) an outer surface adapted to mate with a second vertebral body, and
ii) an inner surface comprising a second articulation surface,
   wherein the first and second articulation surfaces are oriented produce an articulation interface.

Preferably, the articulation interfaces form partial spheres.

In some two piece designs, the second prosthetic endplate can comprise a metal component comprising the outer surface adapted to mate with a second vertebral body, and a polyethylene component comprising the inner surface comprising a second articulation surface. In some embodiments thereof, the polyethylene component could be part of the adjustable component.

In some embodiments, the motion disc does not have an articulating interface. In some embodiments thereof, the motion disc is a cushion-type design having a pair of rigid endplates and a flexible center portion attached thereto. One of the endplates of this embodiment can be provided with a wedge or cam to help adjust the angle or height of the disc. In other embodiments lacking an articulating interface, the motion disc has upper and lower surfaces that articulate with the opposing natural endplates (such as a football-type design). A wedge or cam can be interpositioned between upper and lower pieces of the football-type disc to help adjust the angle or height of the disc.

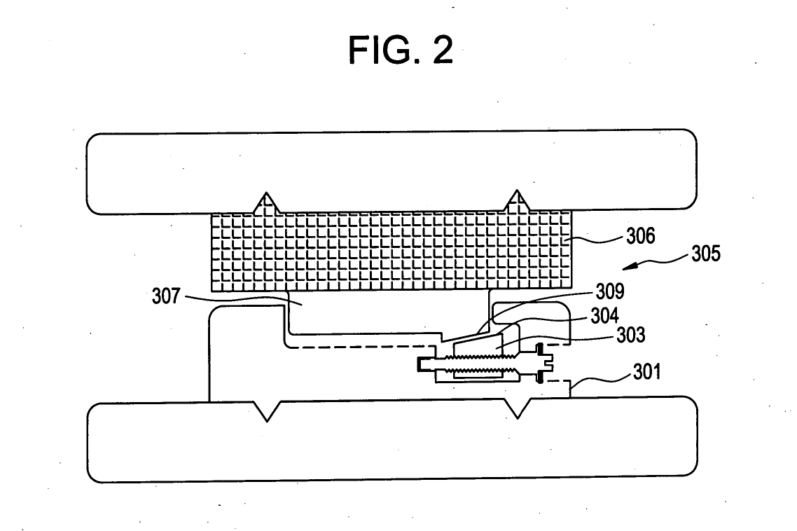

In still other embodiments, and now referring to FIG. 2, the motion disc comprises:
a) a first rigid endplate 301 having a moveable wedge 303 therein, the wedge having an outer surface 304, and
b) a second flexible endplate 305 having a flexible portion 306 attached to a rigid portion 307 having a wedge surface 309,
wherein the outer surface of the wedge of the first rigid endplate slides against the wedge surface of the second flexible endplate.

The motion discs of the present invention can be adapted for use any of the lumbar, thoracic or cervical spine regions. In some embodiments wherein the motion disc is adapted for use in the lumbar region, the three-piece design having a core is selected. In some embodiments wherein the motion disc is adapted for use in the cervical region, the two-piece design is selected.

Preferred articulating motion devices are disclosed in U.S. Pat. Nos. 5,556,431 and 5,674,296, the specifications of which are incorporated by reference. In preferred embodiments thereof, the articulation surface is made of a material selected from the group consisting of a metallic material (such as a titanium alloy, cobalt chromium and stainless steel), and a ceramic material (such as alumina, zirconia and mixtures thereof). Preferably, the core component is adapted for articulation (and so preferably has a surface roughness Ra of no more than 50 um) and more preferably is made of polyethylene, more preferably high molecular weight polyethylene.

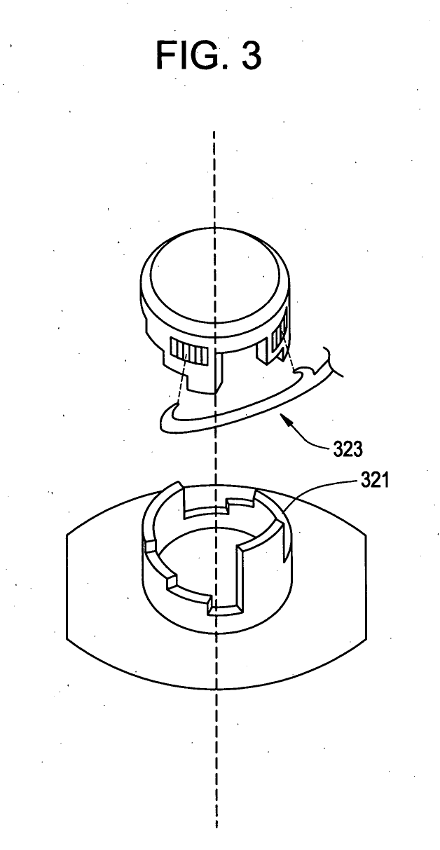

Now referring to FIG. 3, in some embodiments having a more discrete adjustment means, the adjustable endplate of FIG. 1 is modified to possess a cam-type follower adjustment means with stops. In some embodiments thereof, a cam mechanism 321 is coupled with a pawl mechanism 323 to provide positive stops so that an actuation of a magnetic drive advances the rotation to a single predetermined position, thereby providing a single increment of increased height. Since this device would remain fixed in that position unless further activated by a second magnetic force, this embodiment provides the additional benefit of locking capability.

In some embodiments, manual means are employed to drive the adjustment means. This manual means may be carried out by minimally invasive or percutaneous procedures. In some embodiments thereof, the inner plate is associated with a worm and pinion gear adapted to rotate the inner plate upon actuation, thereby providing anti-backlash capabilities.

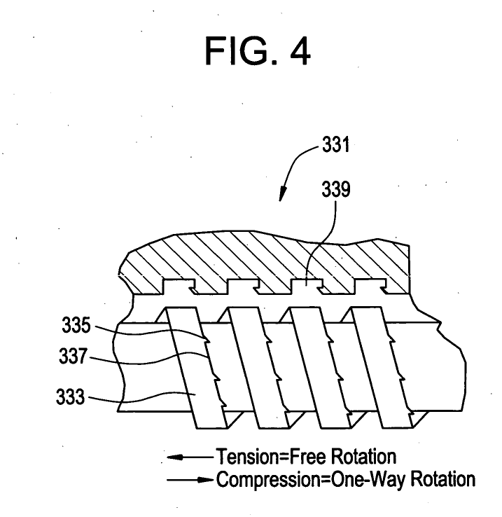

Now referring to FIG. 4, in some embodiments, the adjustment means 331 comprises a thread form 333 that has a ratchet surface 335 on one side 337 of the thread, which would mate with a similar female thread form 339. When this interface is in compression, these mating surfaces would allow only one-way rotation. When this interface is in tension, the device could rotate in either direction since the ratchet teeth would not be engaged.

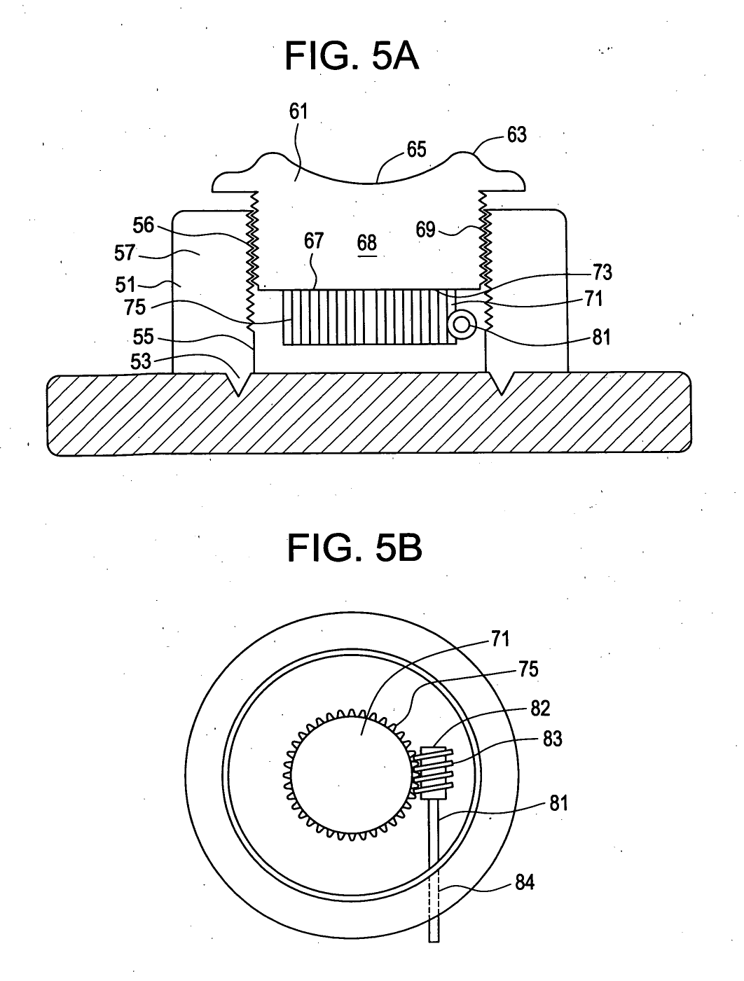

Now referring to FIG. 5a-5b, there is provided a prosthetic endplate in an intervertebral motion disc, the endplate comprising:

i) an outer plate 51 comprising
an outer surface 53 adapted for fixation to a first vertebral body,
an inner surface 55 having a threaded recess 56, and
a body portion 57 therebetween,

ii) an inner plate 61 comprising:
an inner surface 63 having a first articulation surface 65,
   an outer surface 67, and
a body portion 68 therebetween having a threaded lateral surface mated 69 with threaded recess 56 of the outer plate,

iii) a pinion gear 71 having:
an inner surface 73 associated with the outer surface 67 of the inner plate and,
a threaded lateral surface 75,

iv) a worm gear 81 having:
a distal portion 82 having a threaded surface 83 adapted to mate with the threaded lateral surface of the pinion gear 71,
a proximal handle portion 84.
   In use, the worm gear is rotated either manually or by non-invasive means. The non-invasive means may include either rotation of a magnet or motor. When the worm gear is rotated, its rotation causes a corresponding rotation in the pinion gear, which in turn causes a corresponding rotation in the inner plate, thereby causing the inner plate to move away from the vertebral body.

In some embodiments, the angle of the articulation interface (vis-a-vis the natural endplates) may be adjusted. This may be conveniently performed by adding wedge-type components to the adjustment means.

Therefore, in some embodiments, there is provided a prosthetic endplate in an intervertebral motion disc, the endplate comprising:

i) an outer plate comprising
an outer surface adapted for fixation to a first vertebral body,
an inner surface having a recess therein, and
a body portion therebetween, and

ii) an inner plate comprising:
an inner surface having a first articulation surface,
an outer surface having a wedged surface, and
a body portion therebetween disposed within the recess of the outer plate.

In some embodiments, the wedge is oriented to point towards the center of the device, so that moving the wedge towards the center of the device results in increasing the angle of the articulation surface vis-a-vis the natural endplate, while moving the wedge away from the center of the device results in decreasing the angle of the articulation surface vis-a-vis the natural endplate.

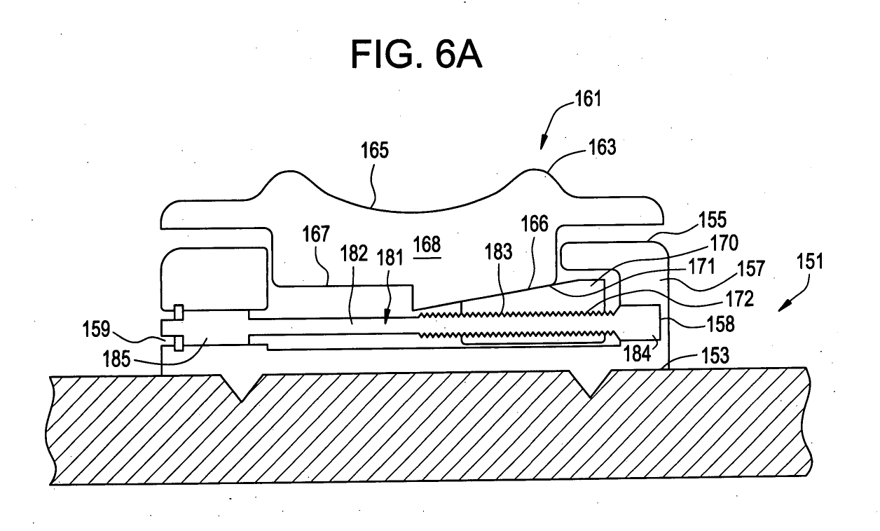

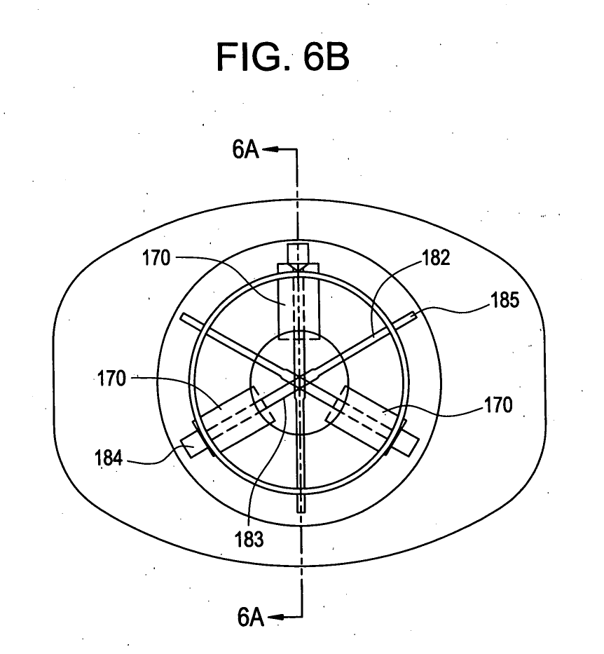

For example, and now referring to FIG. 6a-6b, there is provided a prosthetic endplate in an intervertebral motion disc, the endplate comprising:
i) an outer plate 151 comprising
   an outer surface 153 adapted for fixation to a first vertebral body,
   an inner surface 155 having a recess therein, and
   a body portion 157 therebetween having a first lateral recess 158 and a second lateral recess 159 therein,
ii) an inner plate 161 comprising:
   an inner surface 163 having a first articulation surface 165,
      an outer surface 167 having a wedged surface 166, and
   a body portion 168 therebetween,
iii) a wedge 170 having a corresponding wedge surface 171 for providing sliding contact with the wedged surface 166 of the inner plate and a throughbore 172, and
iv) a captive screw 181 having an elongated shaft 182 having a threaded surface 183 thereon, an enlarged distal head 184, and an enlarged proximal head 185, wherein the elongated shaft is disposed within the throughbore of the wedge 170.

In use, the rotation of the captive screw causes the wedge to move either towards or away from the center of the device, thereby altering the angle of the articulation surface vis-a-vis the natural endplate.

Wedge designs such as that shown in FIG. 6a may be adjusted by either manual or non-invasive techniques. For example, the angle of the device of FIGS. 6a-b is adjusted by rotation of at least one of the proximal heads 185. In such rotation embodiments, proximal head 185 may be adapted to fit a wrench that may be manually rotated. In other rotation embodiments, the proximal head 185 may include a magnet that can be rotated by the rotation of an external magnet.

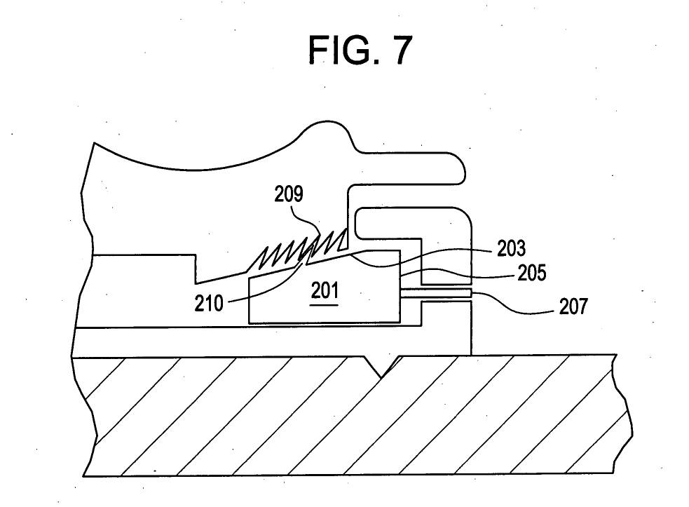

In other embodiments, angle adjustment is effect by translation. Now referring to FIG. 7, there is provided a wedge 201 having a wedged surface 203 for mating with the wedged surface of the inner plate and a proximal surface 205 having a pusher rod 207 attached thereto. In these pusher rod embodiments, angle adjustment is effect by translating the pusher rod. In some embodiments thereof, each of the wedged surfaces are provided with angled teeth 209 for locking in the adjusted angle. In some embodiments, the wedge teeth 210 may be spring loaded to act as a ratchet. A puller or pusher rod may also be used to effect translation.

As shown in FIG. 6b, if the same type of captive screw-wedge assembly is used for all three wedges 170, then access to the socket heads occurs at 120° increments around the device. Accordingly, a surgeon desiring to adjust the angle of the device from each wedge 170 may need to access the device from the anterior side of the patient. Since posterior access to the device is more desirable and less invasive to the patient, there is a need for a device having angle adjustment that may be accessed solely from the posterior side.

Therefore, in some embodiments, there is provided a device similar to that of FIG. 6b, except that one captive screw-and-wedge combination is replaced with the translation-based adjustment means of FIG. 4. In this design, one wedge 170 is accessed from the widened portion of the wedge, and two wedges 170 are accessed from the widened portion of the wedge 170. Thus, the total angle of access can be only 120°, thereby allowing the surgeon to fine tune each wedge from the posterior side of the patient.

As shown in FIGS. 6a-6b, the captive screws traverse essentially the full diameter of the device. Although this provides a simple design, it requires the three screws to be set at different levels of height in order to avoid overlapping at the center of the device. These different heights may undesirably increase the overall height of the device.

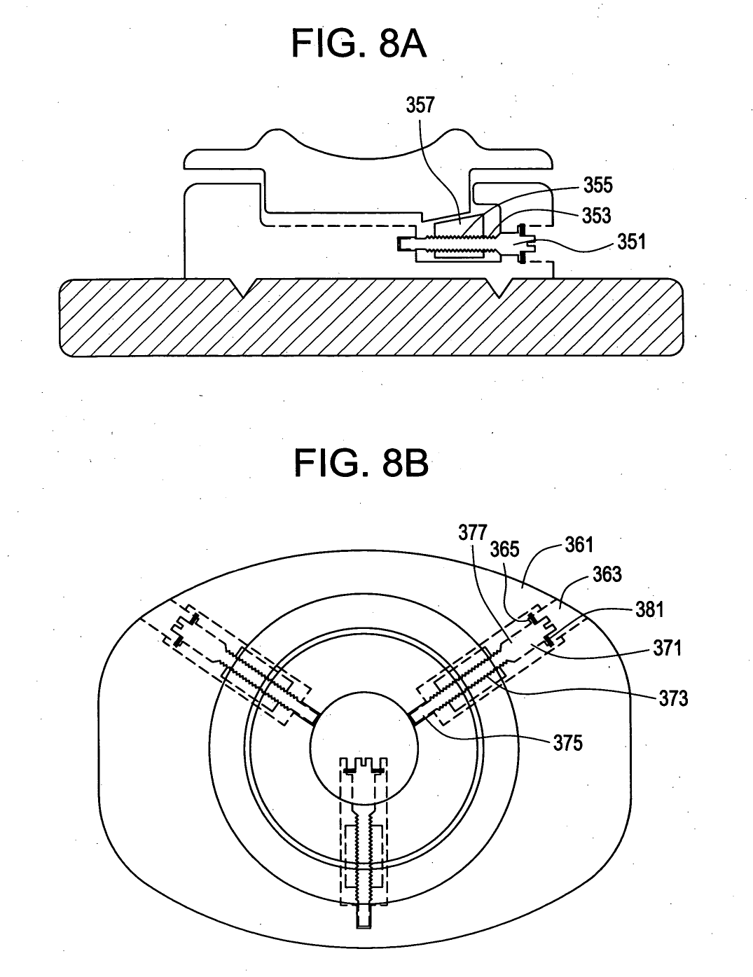

Therefore, in some embodiments, the screw lengths are reduced so they they do not traverse the center of the device. Such as design is shown in FIGS. 8a-8b.

In other embodiments, the wedges of the angle adjustment means are replaced with pneumatic or hydraulic devices. When these expandable devices are filled with fluid and expand, the angle of the device vis-a vis the endplates is increased. When these expandable devices deflate, the angle of the device vis-a vis the endplates is decreased. In some embodiments, the expandable device may be expanded by simply injecting a fluid such as saline or air into the expandable device. In some embodiments, the expandable device may be filled with hygroscopic material that collects water, and thereby expands. In some embodiments, the expandable device may be filled with a chemical fluid that expands in response to an environmental stimulus such as pH.

As shown in FIGS. 8a-b, disposed within lateral recesses of the outer plate is a captured screw 351 having an outer thread 353 adapted to mate with the threaded bore 355 of the wedge 357. This screw comprises a longitudinal shaft having a thread thereon, a blunt distal tip, and a proximal head having a slot. Since both the blunt distal tip and the head ends of the captured screw are respectively seated in an anterior recess and a posterior recess defined by necks of the outer plate, the capture renders the captured screw spatially fixed (save rotation). Rotation of the captured screw bites into the threaded bore of the wedge (which is not fixed), thereby causing relative movement of the wedge to slide relative to the inner plate, thereby affecting the angle of the endplate.

In preferred embodiments, the screws are captured so that they are contained within the outer plate and are limited to rotational movement only.

In the particular embodiment of FIG. 8b, there is provided:
a) an outer plate portion 361 having an inner surface having a longitudinal channel 363 therein, the channel having an first end portion forming a first shoulder 365,
b) a captured screw 371 having a longitudinal shaft having a thread 373 thereon, a first end portion 375 having a blunt tip, and a second end portion 377 having a circumferential projection; and
c) an annular washer 381 disposed about the shaft of the screw and abutting the first shoulder.

In this embodiment, capture of the screw is achieved by providing anterior and posterior shoulder on the mating plate. Anterior movement of the screw will cause annular clip to contact the anterior shoulder (thereby preventing movement in the anterior direction). Posterior movement of the screw will cause the posterior circumferential projection to contact the posterior shoulder (thereby preventing movement in the posterior direction). In some embodiments, a circle clip replaces the snap ring.

In some embodiments, the captured screw comprises a head selected from the group consisting of a slotted head, an Allen head, a Torx® head, a Phillips head, and a Robertson® head.

Although the above devices are suitably used for treatment of degenerative disc disease, in some embodiments, the wedged discs may also be advantageously used to treat spinal deformity. Surgical correction of spinal deformity typically requires fusion of the operated motion segments, severely reducing the flexibility of the spine. Although conventional artificial disc implants are designed to maintain the motion of the spine, they do not conventionally facilitate correction of a deformed spine. Conventional vertebral body tethering can preserve spinal motion, but requires high forces to achieve intraoperative correction.

In some embodiments, the wedged devices of the present invention may be used to correct spinal deformity.

In some embodiments of the present invention, there is provided a a method and device for correcting spinal deformity comprising removing a portion or all of one more intervertebral discs of a deformed spine, then inserting a wedged prosthetic disc designed to correct the spinal deformity while maintaining a majority of the normal spinal range of motion. In another embodiment, the wedged prosthetic disc is designed to act in concert with a vertebral body tether to correct the spinal deformity. The tethers (rods) could also have adjustable height (length).

The method of this invention provides for correction of spinal deformity using an appropriately designed wedged intervertebral disc prosthesis optionally in concert with a vertebral body tether. The wedged prosthetic disc is designed to maintain surgical correction of the deformity while maintaining most of the normal range of motion of the motion segment.

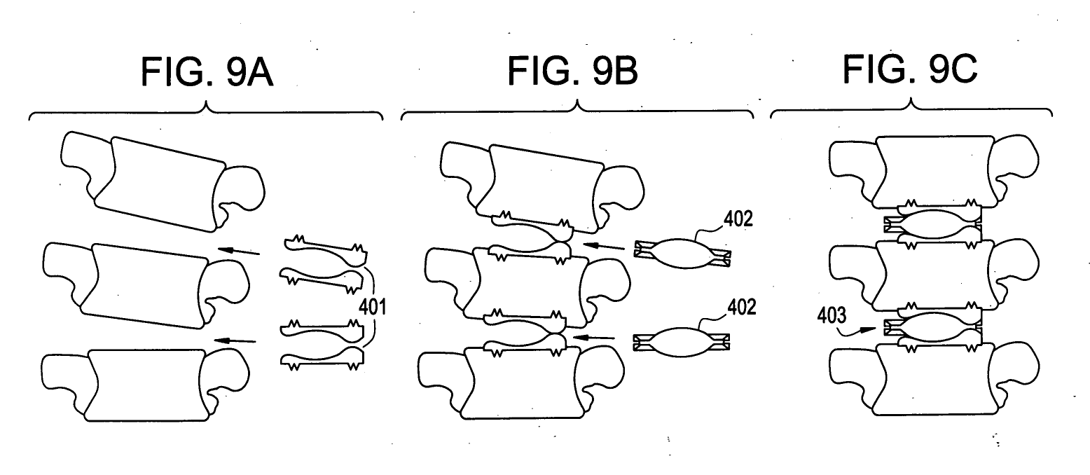

In one embodiment of this invention, a prosthetic disc alone is used to correct the deformed spinal segment(s) as shown in FIGS. 9 a-c. According to the method of the invention, the concave aspect of the spinal deformity is exposed from an antero-lateral approach. The intervertebral disc is then removed from the deformed segments, optionally leaving a portion of the outer annulus in place to stabilize the prosthetic disc. Next, a pair of opposed endplates 401 are inserted antero-laterally into the disc space. Then, a core 402 is inserted antero-laterally between the inserted endplates 401. The prosthetic disc 403 (comprising endplates 401 and core 402) is preferably configured to restrict lateral bending motion of the spinal segment in the direction of the concavity of the deformity. Furthermore, the height of the prosthetic disc is selected such that upon correction of the segment deformity, the prosthetic disc will fill the entirety of the disc space height.

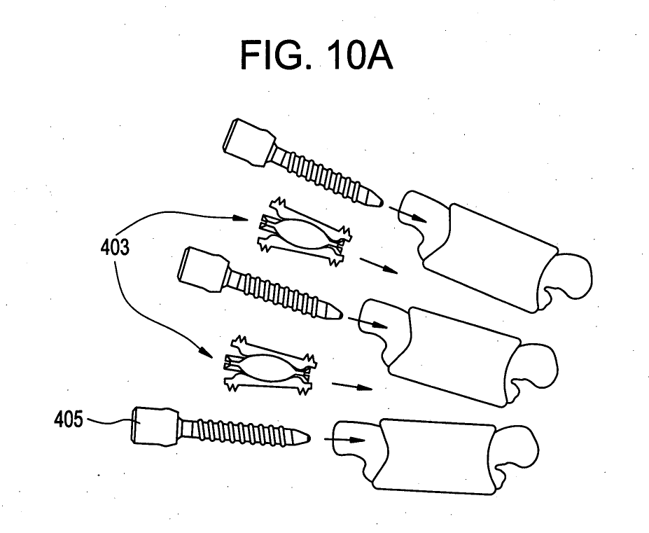

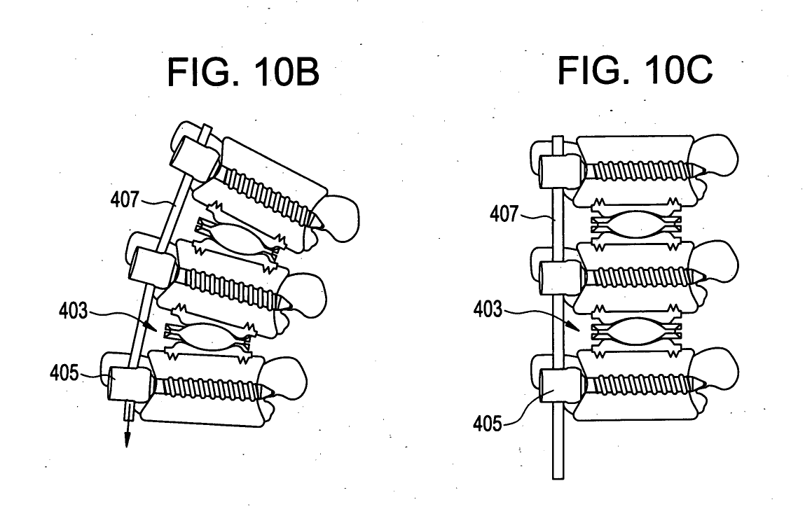

In another embodiment of the method of this invention, the prosthetic disc is used in concert with a vertebral body tether to correct the spinal deformity (FIGS. 10a-c). In this embodiment, the convex aspect of the spinal deformity is exposed from an antero-lateral approach. The intervertebral disc is removed as previously described. The prosthetic disc 403 may be any design known in the art such as articulating designs, spring designs and cushion designs. Preferably, the prosthetic disc is an articulating design. More preferably, the prosthetic disc articulates in an arcing motion with respect to a center of rotation of the prosthetic disc. Upon implantation, the articulating prosthetic disc is configured to fit into the deformed spinal segment. Then, the spinal segment is corrected by articulating the prosthetic disc about its rotation center. Next, spinal fixation elements 405 (such as pedicle screws) having transverse throughholes (not shown) are inserted. Preferably, a flexible vertebral body tether 407 is used to facilitate rotation of the prosthetic disc to correct the deformed segment (FIG. 10b). Then, the tether is preferably fixed through the fixation element throughholes with the spinal segment in its corrected position to maintain the correction (FIG. 10c). In a more preferred embodiment, the flexible tether is made from an absorbable material such that after the spinal elements have adjusted to the corrected orientation, the tether loses strength and disappears, allowing full motion of the spinal segment.

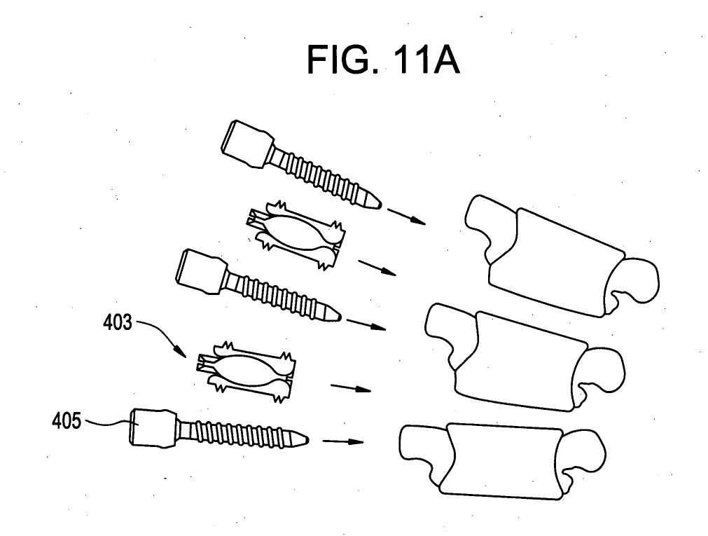

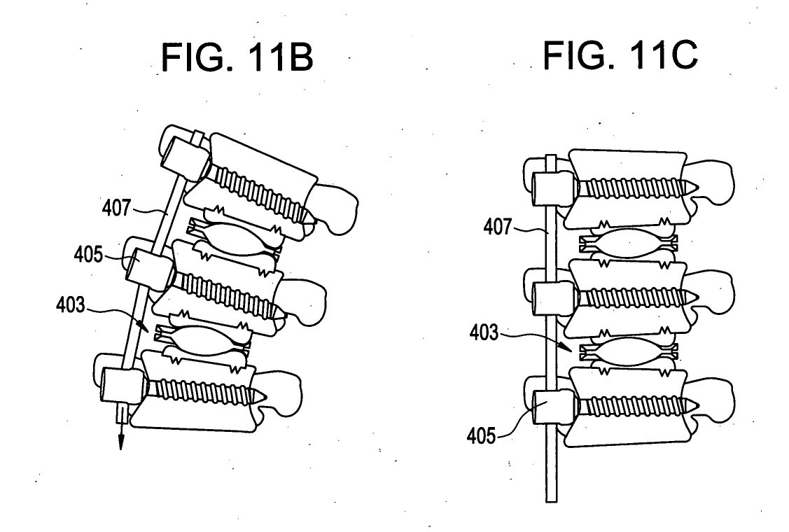

The aforementioned illustrations describe correcting spinal segments in which the intervertebral disc is the primary cause for the deformity. However, in some cases the vertebral bodies are the primary cause for the deformity, exhibiting a wedged appearance. In such a case, the prosthetic disc is preferably configured to accommodate a wedged configuration upon correction of the spinal segment, as illustrated in FIGS. 11 a-c. These illustrations describe a prosthetic disc that would be appropriate for use with a vertebral body tether. However, a prosthetic disc alone can also be used similar to the illustrations provided in FIGS. 9 a-c by increasing the wedging of the disc provided in those figures.

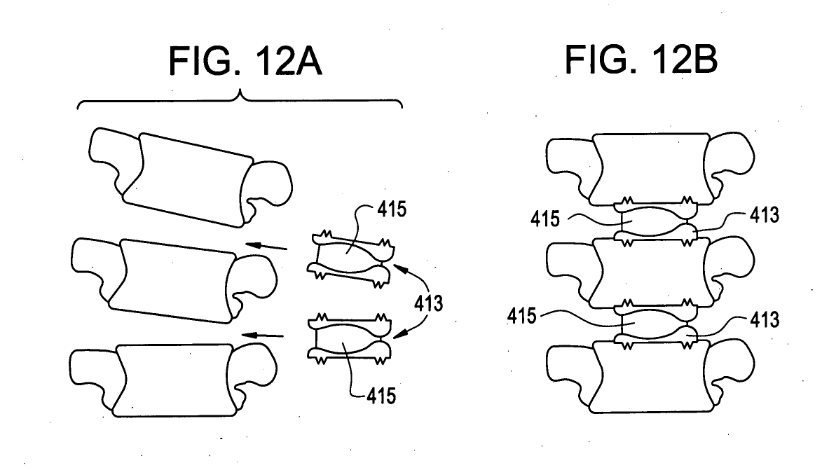

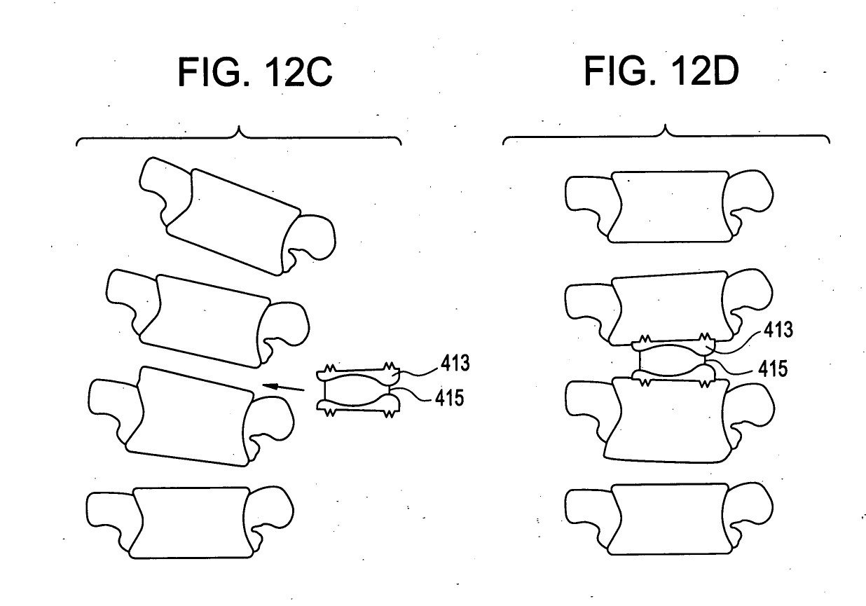

Now referring to FIGS. 12a-b, in some embodiments, a prosthetic disc by itself is used to correct the deformed spinal segment. According to the method of the invention, the concave aspect of the spinal deformity is exposed from an antero-lateral approach. The intervertebral disc is then removed from the deformed segments, optionally leaving a portion of the outer annulus in place to stabilize the prosthetic disc. The prosthetic motion 413 disc may be any motion disc known in the art, such as articulating discs, spring discs and cushion discs. In the embodiment shown in FIGS. 12a-b, the disc is a cushion disc, preferably having an elastomeric material 415 interposed between two endplates. The motion disc is configured to restrict lateral bending motion of the spinal segment in the direction of the concavity of the deformity. Furthermore, the height of the motion disc is selected such that upon correction of the segment deformity, the motion disc will fill the entirety of the disc space height.

Now referring to FIGS. 12c-d, in another preferred embodiment of the present invention, the motion disc 413 of FIGS. 12a-b is modified to have an increased wedge angle. In this configuration, the motion disc 413 will over-correct at the implanted level such that correction can be achieved over multiple levels.

We claim:

1. A prosthetic endplate in an intervertebral motion disc, the endplate comprising:
i) an outer plate comprising
an outer surface adapted for fixation to a first vertebral body,
an inner surface having a threaded recess, and
a body portion therebetween,
ii) an inner plate comprising:
an inner surface having a first articulation surface,
an outer surface, and
a body portion therebetween having a threaded lateral surface,
wherein the threaded lateral surface of the body portion of the inner plate is mated with threaded recess of the outer plate.

2. The endplate of claim 1 wherein the at least one of the plates comprises a marker for determining height or angle.

3. The endplate of claim 2 wherein the marker is a Hall Sensor located upon the outer plate.

4. The endplate of claim 2 wherein the marker is radio-opaque.

5. The endplate of claim 1 wherein the inner plate contains a plurality of magnets.

6. The endplate of claim 1 further comprising:
iii) a pinion gear attached to the outer surface of the inner plate.

7. The endplate of claim 6 further comprising:
iv) a worm gear associated with the pinion gear.

8. The endplate of claim 1 wherein the first articulation surface is convex.

9. The endplate of claim 8 wherein the first articulation surface comprises polyethylene.

10. The endplate of claim 1 wherein the first articulation surface is concave.

11. The endplate of claim 10 wherein the first articulation surface comprises a metal.

12. The endplate of claim 1 further comprising:
iii) discrete adjustment means for discretely adjusting the inner plate relative to the outer plate.

13. The endplate of claim 1 further comprising:
iii) manual adjustment means for manually adjusting the inner plate relative to the outer plate.

14. The endplate of claim 13 wherein the manual adjustment means comprises a pinion gear.

15. The endplate of claim 1 further comprising:
iii) non-invasive adjustment means for non-invasively adjusting the inner plate relative to the outer plate.

16. The endplate of claim 15 wherein the non-invasive adjustment means comprises a magnet.

17. The endplate of claim 1 further comprising:
iii) one-way adjustment means for adjusting the inner plate relative to the outer plate in one direction.

18. A method of adjusting a position of a prosthetic endplate, comprising the steps of:
a) providing a prosthetic endplate comprising:
i) an outer plate comprising an outer surface adapted for fixation to a first vertebral body,
an inner surface having a threaded recess, and
a body portion therebetween,
ii) an inner plate comprising:
an inner surface,
an outer surface, and
a body portion therebetween having a threaded lateral surface,
wherein the threaded lateral surface of the body portion of the inner plate is mated with threaded recess of the outer plate,
b) fixing the outer surface of the outer plate to the first vertebral body to produce a first relative position of the inner plate upon the outer plate, and
c) selectively adjusting the first relative position to a second relative position of the inner plate upon the outer plate.

19. A prosthetic endplate in an intervertebral motion disc, the endplate comprising:
i) an outer plate comprising
an outer surface adapted for fixation to a first vertebral body,
an inner surface having a threaded recess, and
a body portion therebetween,
ii) an inner plate comprising:
an inner surface,
an outer surface, and
a body portion therebetween having a threaded lateral surface,
wherein the threaded lateral surface of the body portion of the inner plate is mated with threaded recess of the outer plate.

20. The endplate of claim 19 wherein the inner surface of the inner plate is non-articulating.

21. A prosthetic endplate in an intervertebral motion disc, the endplate comprising:
i) an outer plate comprising
an outer surface adapted for fixation to a first vertebral body,
an inner surface having a recess therein, and
a body portion therebetween, and
ii) an inner plate comprising:
an inner surface having a first articulation surface,
an outer surface having a wedged surface, and
a body portion therebetween disposed within the recess of the outer plate.

22. The endplate of claim 21 further comprising:
iii) a first wedge having a corresponding wedge surface for providing sliding contact with the wedged surface of the inner plate.

23. The endplate of claim 22 wherein the wedge comprises a throughbore, and
wherein the endplate further comprises:
iv) a captive screw having an elongated shaft having a threaded surface thereon, wherein the elongated shaft is disposed within the throughbore of the wedge.

24. The endplate of claim 22 further comprising:
iv) manual adjustment means for adjusting the wedge relative to the wedge surface.

25. The endplate of claim 24 wherein the manual adjustment means comprises a proximal head located on the captive screw.

26. The endplate of claim 22 further comprising:
iv) non-invasive adjustment means for adjusting the wedge relative to the wedge surface.

27. The endplate of claim 26 wherein the non-invasive adjustment means comprises a proximal head located on the captive screw, wherein the proximal head is magnetic.

28. The endplate of claim 22 wherein the wedge has a pusher rod or puller rod attached thereto.

29. The endplate of claim 22 wherein the wedge comprises at least one tooth.

30. The endplate of claim 29 wherein the wedge comprises a spring-loaded tooth.

31. The endplate of claim 22 wherein the wedged surface comprises a plurality of teeth.

32. The endplate of claim 22 further comprising a second wedge.

33. The endplate of claim 22 further comprising second and third wedges.

34. The endplate of claim 33 wherein the three wedges are spaced about 120 degrees apart.

35. The endplate of claim 33 wherein the first wedge is associated with a pusher rod or puller rod, and the second wedge is associated with a captive screw.

36. The endplate of claim 21 further comprising:
iii) an expandable bag having a corresponding wedge surface for providing contact with the wedged surface of the inner plate.

37. A kit for correcting spinal deformity comprising:
a) an intervertebral motion disc, and
b) a vertebral tether.

38. The kit of claim 37 wherein the motion disc comprises wedged endplates.

39. The kit of claim 37 wherein the motion disc comprises a prosthetic endplate comprising:
i) an outer plate comprising:
an outer surface adapted for fixation to a first vertebral body,
an inner surface having a recess therein, and
a body portion therebetween, and
ii) an inner plate comprising:
an inner surface having a first articulation surface,
an outer surface having a wedged surface, and
a body portion therebetween disposed within the recess of the outer plate.

40. A method of correcting a spinal deformity, comprising the steps of:
a) providing a deformed functional spinal unit comprising an intervertebral disc and opposing endplates,
b) removing at least a portion of the intervertbral disc to form a disc space,
c) inserting an intervertebral motion disc having wedged shape into the disc space.

41. The method of claim 40 wherein the motion disc is inserted into a convex side of a deformity.

42. The method of claim 41 further comprising the step of:
d) tethering the functional spinal unit to correct the deformity.

43. The method of claim 40 wherein the motion disc is inserted into a concave side of a deformity.

44. The method of claim 40 wherein the deformed functional spinal unit comprises deformed opposing endplates.

45. The method of claim 44 further comprising the step of:
d) tethering the functional spinal unit to correct the deformity.

46. A prosthetic endplate in an intervertebral motion disc, the endplate comprising:
i) an outer plate comprising
an outer surface adapted for fixation to a first vertebral body,
an inner surface having a recess therein, and
a body portion therebetween, and
ii) an inner plate comprising:
an inner surface and an outer surface forming a wedge, and
a body portion therebetween disposed within the recess of the outer plate.

47. The endplate of claim 46 wherein the at least one of the plates comprises a marker for determining height or angle.

48. A motion disc comprising:
a) a first rigid endplate having a moveable wedge therein, the wedge having an outer surface, and
b) a second flexible endplate having a flexible portion attached to a rigid portion having a wedge surface,
wherein the outer surface of the wedge of the first rigid endplate slides against the wedge surface of the second flexible endplate.
